SEQUENCE LISTING (7 sequences)

SEQ ID NO: 1 - RNA, 23 nt, Artificial
  synthetic hsa-miR-135b microRNA
uauggcuuuucauuccuauguga

SEQ ID NO: 2 - RNA, 23 nt, Artificial
  synthetic hsa-miR-221 microRNA
agcuacauugucugcuggguuuc

SEQ ID NO: 3 - RNA, 23 nt, Artificial
  synthetic hsa-miR-18a microRNA
uaaggugcaucuagugcagauag

SEQ ID NO: 4 - RNA, 23 nt, Artificial
  synthetic hsa-miR-19a microRNA
ugugcaaaucuaugcaaaacuga

SEQ ID NO: 5 - RNA, 22 nt, Artificial
  synthetic hsa-miR-223 microRNA
ugucaguuugucaaauacccca

SEQ ID NO: 6 - RNA, 23 nt, Artificial
  synthetic hsa-miR-301a microRNA
cagugcaauaguauugucaaagc

SEQ ID NO: 7 - RNA, 22 nt, Artificial
  synthetic hsa-miR-592 microRNA
uugugucaauaugcgaugaugu

(19) United States
(12) Patent Application Publication
(10) Pub. No.: US 2011/0318742 A1
(43) Pub. Date: Dec. 29, 2011

(54) MICRO RNA MARKERS FOR COLORECTAL CANCER

(75) Inventors: Joseph Jao Yiu Sung, Ma On Shan (HK); Jun Yu, Ma On Shan NT (HK); Chung Wah Wu, Lantau (HK)

(73) Assignee: The Chinese University of Hong Kong, Sha Tin (HK)

(21) Appl. No.: 13/166,461

(22) Filed: Jun. 22, 2011

Related U.S. Application Data
(60) Provisional application No. 61/357,818, filed on Jun. 23, 2010.

(51) Int. Cl. C12Q 1/68; C07H 21/02

(52) U.S. Cl. 435/6.11; 435/6.14; 435/6.12; 536/24.33; 536/24.31

(57) ABSTRACT

There are disclosed methods for diagnosing or providing a prognosis for colorectal cancer cells in a biological sample, the method comprising the steps of detecting the presence in the biological sample of an RNA sequence at least about 98% similar over the full sequence length to a sequence selected from the group consisting of SEQ ID NOS:1-7, wherein the presence of the RNA sequence is indicative of the presence of colorectal cancer cells in the sample. Probes for detecting and providing a prognosis for colorectal cancer are also disclosed.

### CROSS-REFERENCES TO RELATED APPLICATIONS

The present application claims benefit under 35 USC 119(e) of U.S. provisional Application No. 61/357,818, filed on Jun. 23, 2010, entitled “Micro-RNA Markers for Colorectal Cancer,” the content of which is incorporated herein by reference in its entirety.

### BACKGROUND OF THE INVENTION

The subject matter disclosed generally relates to markers for colorectal cancer, and methods for detecting colorectal cancer.

It is known that some microRNAs have altered expression in some tumours. The following prior art publications are noted:
Ahmed et. al. “Diagnostic MicroRNA Markers for Screening Sporadic Human Colon Cancer and Active Ulcerative Colitis in Stool and Tissue” Cancer Genomics and Proteomics 6:281-296 (2009)
WO 2007/081470 MICRO-RNA BASED METHODS AND COMPOSITIONS FOR THE DIAGNOSIS AND TREATMENT OF SOLID CANCERS, Croce et. al, filed on Jan. 3, 2007

### BRIEF SUMMARY OF THE INVENTION

In an embodiment there is disclosed a method for diagnosing or providing a prognosis for colorectal cancer cells in a biological sample, the method comprising the steps of detecting the presence in the biological sample of an RNA sequence at least about 98% similar over the full sequence length to a sequence selected from the group consisting of SEQ ID NOS:1-7, wherein the presence of the RNA sequence is indicative of the presence of colorectal cancer cells in the sample.

In alternative embodiments the method may further comprise comparing the level of the sequence in the sample to the level of the sequence in a control.

In alternative embodiments the similarity may be at least about 99% over the full sequence length.

In alternative embodiments the similarity may be about 100% over the full sequence length.

In alternative embodiments the method may further comprise detecting at least two said sequences.

In alternative embodiments the method may further comprise detecting at least four said sequences.

In alternative embodiments the biological sample may be a stool sample.

In alternative embodiments the group may consist of SEQ ID NOS:4-7.

In alternative embodiments the detecting may comprise amplifying the sequence.

In alternative embodiments there is disclosed a method for diagnosing or providing a prognosis for colorectal cancer in a biological sample, the method comprising detecting in the sample an RNA sequence that hybridises under high stringency conditions with a sequence selected from the group consisting of SEQ ID NOS:1-7, wherein the presence of the RNA sequence is indicative of the presence of colorectal cancer cells in the sample.

In alternative embodiments the method may comprising comparing the level of said sequence in said sample to the level of the sequence as in control.

In alternative embodiments the method may further comprise detecting at least two of the sequences or may comprise detecting at least four said sequences.

In alternative embodiments the sample may be a stool sample.

In alternative embodiments the group may consist of SEQ ID NOS:4-7.

In alternative embodiments there is disclosed a kit for diagnosing or providing a prognosis for colorectal cancer cells in a biological sample, the kit comprising:

a primer suitable to reverse transcribe an RNA with at least about 98% similarity over at least 15 contiguous base pairs to any one of SEQ ID NOS:1-7.

In alternative embodiments of the kit, the sequence similarity may be about 100% over the full length of the sequence.

In alternative embodiments of the kit the biological sample may be a stool sample.

In alternative embodiments of the kit, the reverse transcription generates a DNA sequence and the kit further comprises a primer pair suitable to amplify the reverse transcribed DNA sequence.

In alternative embodiments there is disclosed a probe for diagnosing or providing a prognosis for colorectal cancer in a biological sample, the probe comprising a sequence selected from the group consisting of SEQ ID NOS:1-7, wherein the sequence hybridises under high stringency conditions with a RNA sequence present in the sample, and wherein the presence of the RNA sequence is indicative of the presence of colorectal cancer cells in the sample.

### BRIEF DESCRIPTION OF THE DRAWINGS

FIG. 1A is a graphical representation of miR-221 (SEQ ID NO:2) abundance in stool from control and CRC affected subjects.

FIG. 1B is a ROC analysis of miR-221 (SEQ ID NO:2) in stool samples from CRC patients and a control.

FIG. 2A is a graphical representation of miR-135b (SEQ ID NO:2) abundance in stool from control and CRC affected subjects.

FIG. 2B is a ROC analysis of miR-135b (SEQ ID NO:1) in stool samples from CRC patients and a control.

FIG. 3A is a graphical representation of miR-18a (SEQ ID NO:3) abundance in stool from control and CRC affected subjects.

FIG. 3B is a ROC analysis of miR-18a (SEQ ID NO:3) in stool samples from CRC patients and a control.

FIG. 4A is a graphical representation of miR-19a (SEQ ID NO:4) abundance in stool from control and CRC affected subjects.

FIG. 4B is a ROC analysis of miR-19a (SEQ ID NO:4) in stool samples from CRC patients and a control.

FIG. 5A is a graphical representation of miR-223 (SEQ ID NO:5) abundance in stool from control and CRC affected subjects.

FIG. 5B is a ROC analysis of miR-223 (SEQ ID NO:5) in stool samples from CRC patients and a control.

FIG. 6A is a graphical representation of miR-301a (SEQ ID NO:6) abundance in stool from control and CRC affected subjects.

FIG. 6B is a ROC analysis of miR-301a (SEQ ID NO:6) in stool samples from CRC patients and a control.

FIG. 7A is a graphical representation of miR-592 (SEQ ID NO:7) abundance in stool from control and CRC affected subjects.

FIG. 7B is a ROC analysis of miR-592 (SEQ ID NO:7) in stool samples from CRC patients and a control.

FIG. 8 shows a series of human miRNA sequences according to embodiments (SEQ ID NOS:1-7).

FIG. 9 is a table of parameters for ROC analysis and diagnostic analysis in embodiments.

### DETAILED DESCRIPTION OF THE INVENTION

### Terms

In this disclosure the following terms have the meanings set forth below:

In this disclosure the term “or” is generally employed in its sense including “and/or” unless the content clearly dictates otherwise.

In this disclosure, the term “biomarker” or “marker” means a substance such as a gene, or a measurement of a variable related to a disease that may serve as an indicator or predictor of that disease. Biomarkers or markers are parameters from which the presence or risk of a disease can be inferred, and in embodiments may be used determine a prognosis or provide a measure of a disease.

In this disclosure the terms “nucleic acid”, “nucleic acid sequence,” and the like mean polynucleotides, which may be gDNA, cDNA or RNA and which may be single-stranded or double-stranded. The term also includes peptide nucleic acid (PNA), or any chemically DNA-like or RNA-like material. “cDNA” refers to copy DNA made from mRNA that is naturally occurring in a cell. “gDNA” refers to genomic DNA. Combinations of the same are also possible (i.e., a recombinant nucleic acid that is part gDNA and part cDNA).

In this disclosure the term “microRNA” or “miRNA” or the equivalent, means short RNA sequences, which may be between about 15 and 30 nucleotides long (or longer or shorter) and which may be non-coding.

In this disclosure the term “ROC” means “receiver operating characteristic” and refers to a method of nucleic acid analysis. A ROC analysis may be used to evaluate the diagnostic performance of a test. A ROC graph is a plot of sensitivity % and specificity % of a test at various cut-off values. An ROC curve may be used to differentiate between two sample groups, such as a control or normal sample having specified characteristics, and a test or experimental sample. Usually the distributions seen in the two samples will overlap, making it a non-trivial exercise to determine whether there is a real difference between them. If the discrimination threshold or specificity of a ROC analysis is set high, then the test is less likely to generate a false positive, ie. less likely wrongly identify a difference between the two samples. However, in these circumstances the test will be more likely to miss instances where there is a real difference between the samples and consequently it is more likely that some cases of disease will not be identified. If the sensitivity of the test is increased, there is a corresponding fall in specificity. Thus if the test is made more sensitive then the test is more likely to identify most or all of the people with the disease, but will also diagnose the disease in more people who do not have it.

Each point on a ROC curve represents the sensitivity and its respective specificity. A cut-off value can be selected based on an ROC curve to identify a point where sensitivity and specificity both have acceptable values, and this point can be used in applying the test for diagnostic purposes. While a user is able to modify the parameters in ways that will be readily understood by those skilled in the art, for the examples described in this disclosure each threshold was chosen to obtain both reasonable sensitivity and specificity. In particular instances both of these were maintained at approximately 60% to 90%, although lower and higher values are possible.

Another useful feature of the ROC is the area under curve (AUC) value, which quantifies the overall ability of the test to discriminate between different sample properties, in this case to discriminated between those individuals with colorectal cancer and those without colorectal cancer. A test that is no better at identifying true positives than random chance will generate a ROC curve with an area of 0.5. A test having perfect specificity and sensitivity, that generates no false positives and no false negatives, will have an an area of 1.00. In reality, any test will have an area somewhere between these two values.

In one application used herein, a ROC graph may be plotted based on the result of detecting specific microRNAs in stool samples of CRC patients and control individuals to generate a plot of sensitivity % and specificity % of the corresponding stool miRNA test at various cut-off values. The use of ROC analysis will be readily understood and implemented by those skilled in the art The ROC curve was generated using a statistical software (Prism) based on input data of miRNA levels of cancer group and non-cancer group.

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42. ° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are well known in the art and are provided, e.g., in Innis et al. (1990) PCR Protocols, A Guide to Methods and Applications, Academic Press, Inc. N.Y.).

In this disclosure the terms “gene expression” and “protein expression” mean and include any information pertaining to the amount of gene transcript or protein present in a sample, as well as information about the rate at which genes, RNA or proteins are being expressed or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

In this disclosure the term “oligonucleotide” means a molecule comprised of two or more nucleotides, preferably more than three. Its exact size will depend upon many factors which, in turn, depend upon the ultimate function and use of the oligonucleotide. In particular embodiments an oligonucleotide may have a length of about 10 nucleotides to 100 nucleotides or any integer therebetween. In embodiments oligonucleotides may be about 10 to 30 nucleotides long, or may be between about 20 and 25 nucleotides long. In embodiments an oligonucleotide may be greater than about 10, 11, 12, 13, 14, 15, 16, 17, 18, 19, 20, 21, 22, 23, 24, or 25 nucleotides long for specificity. In certain embodiments oligonucleotides shorter than these lengths may be suitable.

In this disclosure the term “primer” means an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA or RNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic and prognostic applications, depending on the complexity of the target sequence, the oligonucleotide primer typically contains at least or more than about 10, or 15, or 20, or 25 or more nucleotides, although it may contain fewer nucleotides or more nucleotides. The factors involved in determining the appropriate length of primer are readily known to one of ordinary skill in the art.

In this disclosure the term “primer pair”, means a pair of primers which hybridize to opposite strands a target DNA molecule, to regions of the target DNA which flank a nucleotide sequence to be amplified.

In this disclosure the term “primer site”, means the area of the target DNA to which a primer hybridizes.

In this disclosure, the nucleic acids described and claimed refer to all forms of nucleic acid sequences, including but not limited to genomic nucleic acids, pre-mRNA, mRNA, miRNA, cDNA, cRNA, polymorphic variants, alleles, mutants, and interspecies homologs that:
(1) specifically hybridize under stringent hybridization conditions to a disclosed nucleic acid sequence or to a nucleic acid sequence encoding a disclosed amino acid sequence, and conservatively modified variants thereof,
(2) have a nucleic acid sequence that has greater than about 95%, preferably greater than about 96%, 97%, 98%, 99%, or 100% nucleotide sequence identity, preferably over a region of at least about 15, 25, 30, 35 40, 50, 100 or more nucleotides, to a reference nucleic acid sequence.

In this disclosure the term “biological sample” or “sample” includes sections or samples of tissues such as biopsy and autopsy samples, and frozen sections and samples taken for histologic purposes, or processed forms of any of such samples. Biological samples include blood and blood fractions or products (e.g., serum, plasma, platelets, red blood cells, and the like), sputum or saliva, lymph and tongue tissue, cultured cells, e.g., primary cultures, explants, and transformed cells, stool, urine, stomach biopsy tissue etc. A biological sample is typically obtained from a eukaryotic organism, which may be a mammal, may be a primate and may be a human subject. Biological samples may be treated and processed for use by any conventional procedures, all of which will be readily understood and implemented by those skilled in the art. In particular alternative embodiments microRNA may be extracted from stool using a variety of methods using suitable reagents. In particular embodiments such reagents may include any of the following, all used following methods described or suggested by the manufacturers, or by others: TRIzol Reagent (Invirogen, Carlsbad, Calif., UCA), TRIzol LS reagent (Invirogen, Carlsbad, Calif., UCA), miRNeasy Mini Kit (Qiagen, Valencia, Calif., USA);. mirVana miRNA Isolation Kit (Applied Biosystems, Foster City, Calif., US); miRCURY RNA Isolation Kits (Exiqon, Vedbaek, Denmark). In particular embodiments, any commercially available kits or reagents suitable to isolate microRNA from biological samples may be useable.

In this disclosure the term “biopsy” refers to the process of removing a tissue sample for diagnostic or prognostic evaluation, and to the tissue specimen itself. Any suitable biopsy technique known in the art can be applied to the diagnostic and prognostic methods of the present invention. The biopsy technique applied will depend on the tissue type to be evaluated (e.g., tongue, colon, prostate, kidney, bladder, lymph node, liver, bone marrow, blood cell, stomach tissue, etc.) among other factors. Representative biopsy techniques include, but are not limited to, excisional biopsy, incisional biopsy, needle biopsy, surgical biopsy, and bone marrow biopsy and may comprise colonoscopy. A wide range of biopsy techniques are well known to those skilled in the art who will choose between them and implement them with minimal experimentation.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA, or genomic library) or a portion of a gel (e.g., agarose, or polyacrylamine) containing restriction-digested genomic DNA is not to be considered an isolated nucleic acid.

In this disclosure a “cell” may be isolated, may be comprised in a group of cells, may be in culture, or may be comprised in a living subject and may be a mammalian cell and may be a human cell. Similarly “tissue” may comprise any number of cells and may be comprised in a living subject or may be isolated therefrom.

In this disclosure “cancer” means and includes any malignancy, or malignant cell division or malignant tumour, or any condition comprising uncontrolled or inappropriate cell proliferation and includes without limitation any disease characterized by uncontrolled or inappropriate cell proliferation.

In this disclosure the terms “colon cancer”, “colorectal cancer” and “CRC” have the same meaning and mean a cancer of the colon, or rectum and include cells characteristic of colorectal cancer. Without limitation, colorectal cancers may be adenocarcinomas, leiomyosarcomas, lymphomas, melanomas, and neuroendocrine tumors and include precancerous cells and groups of cells.

In this disclosure the term “colon cancer cell” or “colorectal cancer cell” or “CRC cell” means a cell characteristic of colon cancer or colorectal cancer, and includes cells which are precancerous.

In this disclosure the term “precancerous” means a cell which is in the early stages of conversion to a cancer cell or which is predisposed for conversion to a cancer cell. Such cells may show one or more phenotypic traits characteristic of the cancerous cell.

In this disclosure the term “purified,” or “substantially purified” or “extracted” when used with reference to nucleic acids or polypeptides, means nucleic acids or polypeptides separated from their natural environment so that they are at least about 50%, 55%, 60%, 65%, 70%, 75%, 80, 85, 90 or 95% of total nucleic acid or polypeptide or organic chemicals in a given sample. Protein purity may be assessed herein by SDS-PAGE and silver staining Nucleic acid purity may be assessed by agarose gel and EtBr staining.

In this disclosure the term “detection” means any process of observing a marker, or a change in a marker (such as for example the change in the methylation state of the marker, or the level of expression of nucleic acid or protein sequences), in a biological sample, whether or not the marker or the change in the marker is actually detected. In other words, the act of probing a sample for a marker or a change in the marker, is a “detection” even if the marker is determined to be not present or below the level of sensitivity. Detection may be a quantitative, semi-quantitative or non-quantitative observation and may be based on a comparison with one or more control samples. It will be understood that detecting a colon cancer as disclosed herein includes detecting precancerous cells that are beginning to or will, or have an increased predisposition to develop into colon cancer cells. Detecting a colon cancer may also include detecting a likely probability of mortality or a likely prognosis for the condition.

In this disclosure the terms “homology”, “identity” and “similarity” mean sequence similarity between two peptides or between two nucleic acid molecules. They can each be determined by comparing a position in each sequence which may be aligned for purposes of comparison. When an equivalent position in the compared sequences is occupied by the same base, then the molecules are identical at that position. Expression as a percentage of homology/similarity or identity refers to a function of the number of identical bases at positions shared by the compared sequences. A sequence which is “unrelated or “non-homologous” shares less than 40% identity, preferably less than 25% identity with a sequence of the present invention. In comparing two nucleic acid sequences, the absence of residues or presence of extra residues also decreases the identity and homology/similarity. In particular embodiments two or more sequences or subsequences may be considered substantially or significantly homologous, similar or identical if their sequences are about 60% identical, or are about 65%, 70%, 75%, 80%, 85%, 90%, 91%, 92%, 93%, 94%, 95%, 96%, 97%, 98%, 99%, or 100% over a specified region, when compared and aligned for maximum correspondence over a comparison window or designated region, as measured using a BLAST or BLAST 2.0 sequence comparison algorithms with default parameters described below, or by manual alignment and visual inspection such as provided on-line by the National Center for Biotechnology Information (NCBI). This definition also refers to, or may be applied to, the compliment of a test sequence. Thus, to the extent the context allows, for instance where a nucleotide sequence may be expected to naturally occur in a DNA duplex, or may naturally occur in the form of either or both of the complementary strands, then a nucleotide sequence that is complimentary to a specified target sequence or its variants, is itself deemed “similar” to the target sequence and a reference to a “similar” nucleic acid sequence includes both the single strand sequence, its complimentary sequence, the double stranded complex of the strands, sequences able to encode the same or similar polypeptide products, and any permissible variants to any of the foregoing. Circumstances where similarity must be limited to an analysis of the sequence of a single nucleic acid strand may include for example the detection and quantification of the expression of a specific RNA sequence or coding sequence within a cell. The definition also includes sequences that have deletions and/or additions, as well as those that have substitutions. In embodiments identity or similarity may exist over a region that is at least about 10, 11, 12, 13, 14, 15, 16, 17, 18, 19, 10, 21, 22, 23, 24, 25 or more amino acids or nucleotides in length, or over a region that is more than about 10, 15, 20, 25, 30, 35, 40, 45, 50, 55, 60, 65, 70, 75, 80, 85, 90, 95 or more than about 100 amino acids or nucleotides in length.

In this disclosure the term “amplify”, means a process whereby multiple copies are made of one particular locus of a nucleic acid, such as genomic DNA or cDNA. Amplification can be accomplished using any one of a number of known means, including but not limited to the polymerase chain reaction (PCR), transcription based amplification and strand displacement amplification (SDA) and may comprise generating a cDNA strand from an RNA template and then amplifying the thus created DNA strand.

In this disclosure the term “polymerase chain reaction” or “PCR”, means, a technique in which cycles of denaturation, annealing with primer, and extension with DNA polymerase are used to amplify the number of copies of a target DNA sequence by approximately 10⁶ times or more. The polymerase chain reaction process for amplifying nucleic acid is covered by U.S. Pat. Nos. 4,683,195 and 4,683,202. Those skilled in the art will readily select and implement suitable primers and primer pairs both to generate cDNA strands where desired, and to amplify desired DNA and RNA sequences as desired.

In this disclosure a “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable.

In this disclosure the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

### Exclusion of Certain Sequences:

It will be understood that in particular embodiments individual examples of sequences, probes, primers, polypeptides or the like may be excluded.

### Detection of Nucleic Acids:

A range of methods for the detection of specific nucleic acid sequences and their application will be readily apparent to those skilled in the art.

Nucleic acid molecules can be detected using a number of different methods. Methods for detecting nucleic acids include, for example, PCR and nucleic acid hybridizations (e.g., Southern blot, Northern blot, or in situ hybridizations). Specifically, oligonucleotides (e.g., oligonucleotide primers) capable of amplifying a target nucleic acid can be used in a PCR reaction. PCR methods generally include the steps of obtaining a sample, isolating nucleic acid (e.g., DNA, RNA, or both) from the sample, and contacting the nucleic acid with one or more oligonucleotide primers that hybridize(s) with specificity to the template nucleic acid under conditions such that amplification of the template nucleic acid occurs. In the presence of a template nucleic acid, an amplification product is produced. Conditions for amplification of a nucleic acid and detection of an amplification product are known to those of skill in the art. A range of modifications to the basic technique of PCR also have been developed, including but not limited to anchor PCR, RACE PCR, RT-PCR, and ligation chain reaction (LCR). A pair of primers in an amplification reaction must anneal to opposite strands of the template nucleic acid, and should be an appropriate distance from one another such that the polymerase can effectively polymerize across the region and such that the amplification product can be readily detected using, for example, electrophoresis. Oligonucleotide primers can be designed using, for example, a computer program such as OLIGO (Molecular Biology Insights Inc., Cascade, Colo.) to assist in designing primers that have similar melting temperatures. Typically, oligonucleotide primers are 10 to 30 or 40 or 50 nucleotides in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length), but can be longer or shorter if appropriate amplification conditions are used. It will be understood that RNA sequences may be detected by generaging a suitable cDNA strand by reverse transcription, and subsequently amplifying or detecting the resulting DNA sequence using suitable methods.

In this disclosure the term “standard amplification conditions” refers to the basic components of an amplification reaction mix, and cycling conditions that include multiple cycles of denaturing the template nucleic acid, annealing the oligonucleotide primers to the template nucleic acid, and extension of the primers by the polymerase to produce an amplification product.

Detection of an amplification product or a hybridization complex is usually accomplished using detectable labels. The term “label” with regard to a nucleic acid is intended to encompass direct labeling of a nucleic acid by coupling (i.e., physically linking) a detectable substance to the nucleic acid, as well as indirect labeling of the nucleic acid by reactivity with another reagent that is directly labeled with a detectable substance. Detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. An example of indirect labeling includes end-labeling a nucleic acid with biotin such that it can be detected with fluorescently labeled streptavidin.

The term “probe” with regard to nucleic acid sequences is used in its ordinary sense to mean a selected nucleic acid sequence that will hybridise under specified conditions to a target sequence and may be used to detect the presence of such target sequence. It will be understood by those skilled in the art that in some instances probes may be also be useable as primers, and primers may useable as probes.

### Articles of Manufacture

This disclosure encompasses articles of manufacture (e.g., kits) that contain one or more nucleic acid molecules, or one or more vectors that encode a nucleic acid molecule. Such nucleic acid molecules are formulated for use as described herein, and can be packaged appropriately either separately or collectively, for the intended mode of use. For example, a nucleic acid molecule or a vector encoding a nucleic acid molecule can be contained within or accompanied by a suitable buffer or suitable labelling reagent or detection system.

Kits according to embodiments can include additional reagents (e.g., buffers, co-factors, enzymes detection system). Kits may also contain a control sample or a series of control samples that can be assayed and compared to the biological sample. Each component of a kit may be enclosed within an individual container and all of the various containers may be within a single package.

### EMBODIMENTS

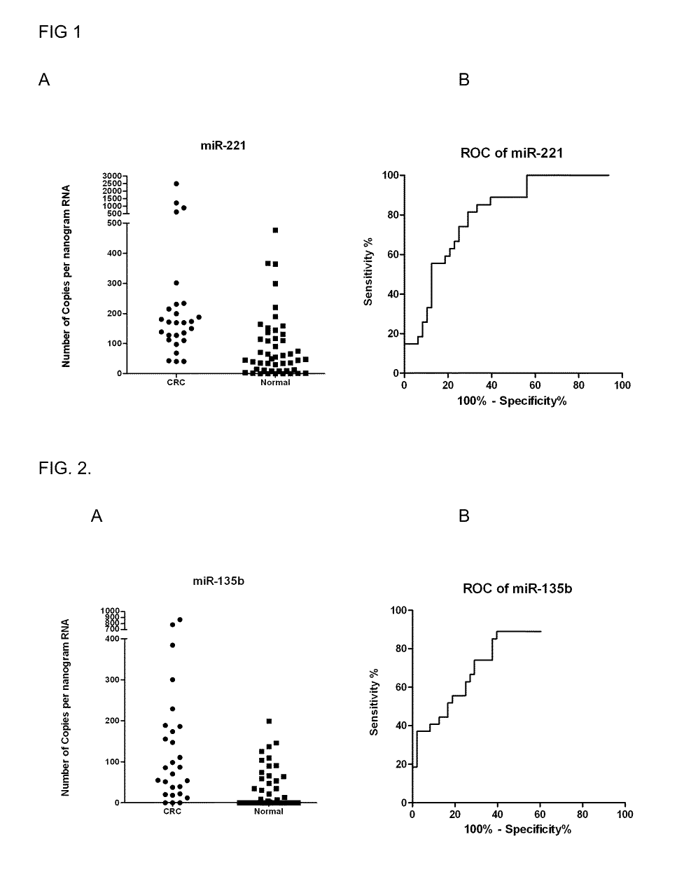

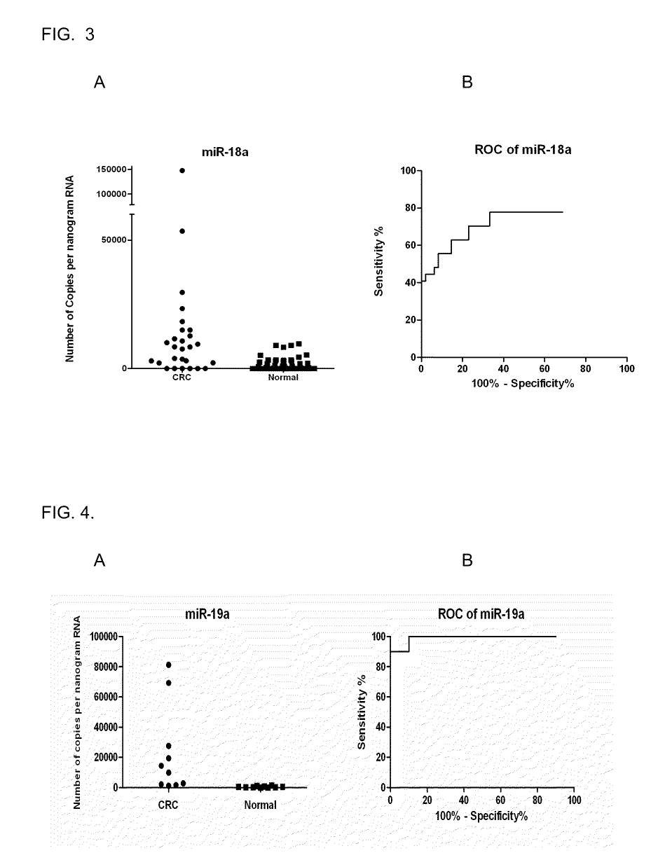

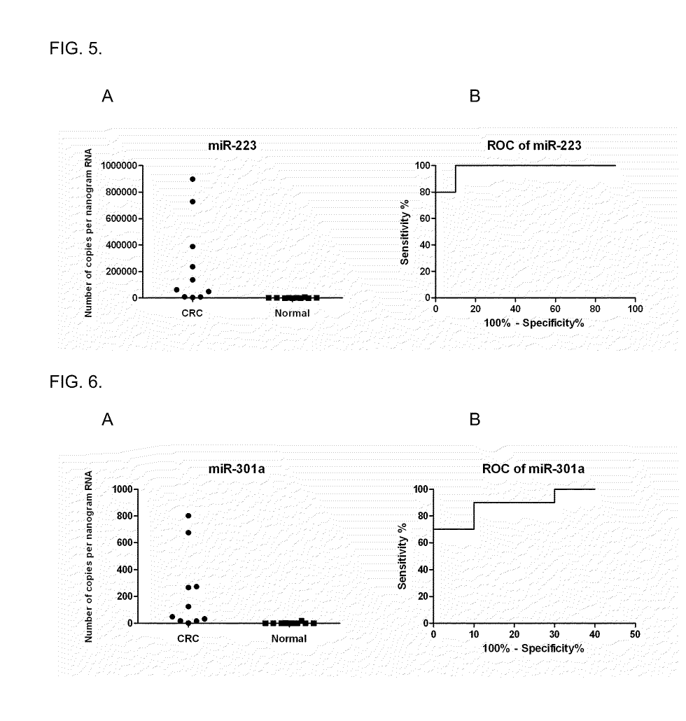

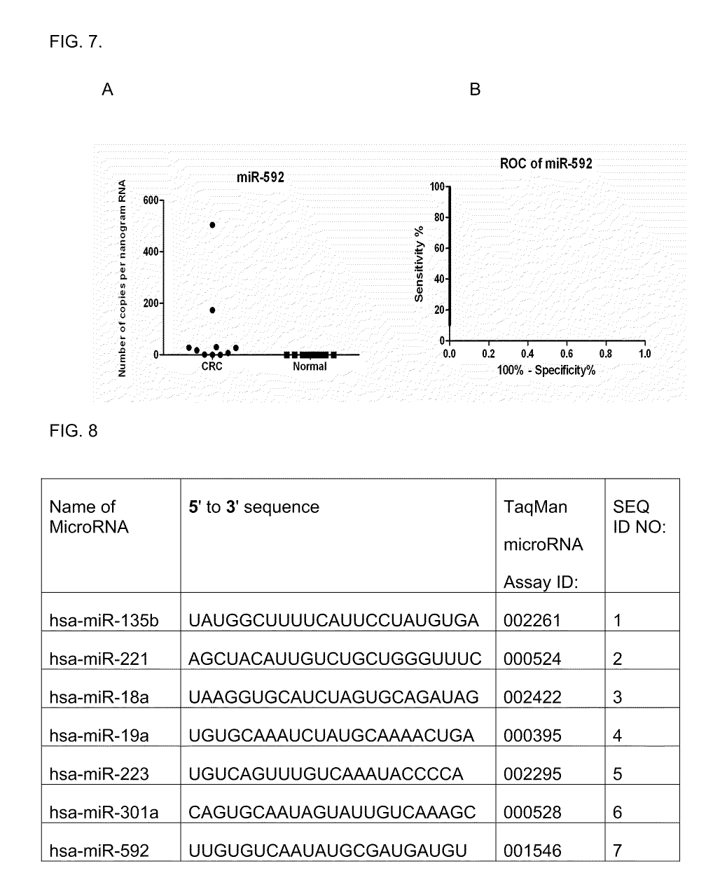

Embodiments of the subject matter claimed are described with general reference to FIGS. 1 through 9.

In a first embodiment there is disclosed a method for diagnosing or providing a prognosis for colorectal cancer in a biological sample. In embodiments the method comprises detecting the presence in the biological sample of an RNA sequence at least about 98% similar over the full sequence length to a sequence selected from the group consisting of SEQ ID NOS:1-7, the sequences and identities of which are further set out in FIG. 8 along with the TaqMan microRNA assays used for the detection of the sequences. In embodiments of the method, an elevated level of the sequence is indicative of the presence of colorectal cancer. The detection of such elevated levels of a sequence may comprise comparison between levels of the sequence in the test sample and a reference level. Such reference level may be determined by comparison with a control, which control may be or may comprise a non-cancerous control sample, or a cancerous control sample, or an artificially generated or extracted sample, or reference standard or may comprise comparing the sequence level in at test sample with predetermined reference levels. In alternative embodiments, the sequence similarity may be at least about 99% or at least about 100% and may be apparent over the full sequence length.

In alternative embodiments, the method may comprise detecting at least one, two, three, four, five, six, seven or eight sequences selected from the group consisting of SEQ ID NOS:1-7. In alternative embodiments the detection of any one or more of SEQ ID NOS:1-7 may be combined with detecting additional sequences or performing additional diagnostic or prognostic tests. In alternative embodiments the levels of one or more of the selected sequences may be quantified or independently or collectively compared with suitable controls.

In particular embodiments, the test sequences may be selected from the group consisting of SEQ ID NOS:1-7. In particular embodiments the biological sample may be taken from a subject, which may be a human subject. In embodiments the biological sample may be a stool sample. In embodiments the detecting may comprise amplifying the relevant sequence or sequences or may comprise any other form of detection methodology, all suitable such methods will be readily understood and implemented by those skilled in the art.

In particular embodiments the amplification of specific microRNA was carried out using TaqMan microRNA Assays (Part Number: 442797, Applied Biosystems, Foster City, Calif., US). The assay number of each microRNA assay is shown in FIG. 8. Each kit includes a primer for reverse transcription of specific microRNA, and primer/probe mixtures for qPCR of that microRNA. The primer and probe sequences are not disclosed by the company, but are known to be highly stringent and specific. Additional data regarding the assay system can be accessed at:https://products.appliedbiosystems.com/ab/en/US/adirect/ab?cmd=catNavigate2&catID=6018 03&tab=DetailInfo.

In particular embodiments, alternative assay systems may be useable to carry out assays, such as kits supplied by Qiagen. In one embodiment suitable assay systems and kits may provide two steps for the analysis. One step may be reverse transcription (RT), to transcribe the short miRNA sequence into a lengthened cDNA sequence by attaching a specific RT primer. The second step in the analysis is quantitative PCR which amplifies the elongated sequence using forward and reverse primer pairs for the added sequences, and a probe to detect the amplified product. Those skilled in the art will understand that many other methodologies may be used to detect and quantify miRNA sequences and it will be understood that any of these may be used with such routine adaptations as may be necessary or desirable. The subject matter disclosed and claimed herein is in no way limited by the use of any specific technical methods for obtaining, detecting, analysing, amplifying, modifying or quantifying miRNA and is likewise not limited by any particular choice of primers or primer sequences.

In alternative embodiments user defined primers can be used and will be readily designed and implemented by those skilled in the art using routine techniques. In alternative embodiments, there is disclosed method for diagnosing or providing a prognosis for colorectal cancer in a biological sample, the method comprising detecting in the sample a sequence that hybridises under high stringency conditions with a sequence selected from the group consisting of SEQ ID NOS:1-7. It will be understood that in variants of the embodiment, the particular stringency used may be adjusted in ways that will be readily apparent to those skilled in the art. It will be understood that in embodiments an elevated level of the sequence in the sample relative to a control may be taken as indicative of the presence of colorectal cancer. In embodiments the method may comprise detecting at least about one, two, three, four, five, six, seven, or eight sequences selected from SEQ ID NOS:1-7. In particular embodiments the sequence may be selected from the group consisting of SEQ ID NOS:1-3. In particular embodiments the sequence may be selected from the groups consisting of SEQ ID NOS:1-3, or SEQ ID NOS:2-7, 3-7, 4-7, 5-7, 6 and 7, or, SEQ ID NOS:1 and 2, 1-3, 1-4, 1-5, 1-6, 1-5, 1-4, 1-3 or 1 and 2. In further embodiments the related sequences may comprise any selected ones or pluralities of ones of SEQ ID NO:1, 2, 3, 4, 5, 6 and 7 and may comprise such sequences in any combination and including or omitting any one or more of such sequences.

In alternative embodiments the methods may be implemented using a kit and accordingly in one embodiment there is disclosed a kit for diagnosing or providing a prognosis for colorectal cancer in a biological sample, the kit comprising: at least two primers suitable to amplify a region of DNA with at least about 98% similarity over at least 15 contiguous base pairs to any one of SEQ ID NOS:1-7. In embodiments the method may comprise detecting at least about one, two, three, four, five, six, seven selected from SEQ ID NOS:1-7. In particular embodiments the sequence may be selected from the groups consisting of SEQ ID NOS:1-3, or SEQ ID NOS:2-7, 3-7, 4-7, 5-7, 6 and 7, or, SEQ ID NOS:1 and 2, 1-3, 1-4, 1-5, 1-6, 1-5, 1-4, 1-3 or 1 and 2. In further embodiments the related sequences may comprise any selected ones or pluralities of ones of SEQ ID NOS:1, 2, 3, 4, 5, 6 and 7 and may comprise such sequences in any combination and including or omitting any one or more of such sequences. In further embodiments sequence similarity may be about 100% over the full length of the sequence. In embodiments the biological sample may be a stool sample.

In particular embodiments of the methods and sequences disclosed herein, the sensitivity of tests for colorectal cancer is at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90, 91%, 92%, 93%, 94%, 95%, 96%, 97%, 98%, 99%, and 100%. In particular embodiments of the methods and sequences disclosed herein the specificity of tests for colorectal cancer is at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90, 91%, 92%, 93%, 94%, 95%, 96%, 97%, 98%, 99%, and 100%.

In particular embodiments of the methods and sequences disclosed herein, the sensitivity of tests for colorectal cancer may be between about 60% and about 90%, between about 60% and about 70%, between about 70% and about 80%, between about 80% and about 90%, between about 90% and about 100%, between about 65% and about 75%, between about 75% and about 85%, between about 85% and about 95%, or between about 95% and about 100%. In particular embodiments of the methods and sequences disclosed herein the specificity of tests for colorectal cancer may be between about 60% and about 90%, between about 60% and about 70%, between about 70% and about 80%, between about 80% and about 90%, between about 90% and about 100%, between about 65% and about 75%, between about 75% and about 85%, between about 85% and about 95%, or between about 95% and about 100%.

In particular embodiments of the methods and sequences disclosed herein, the cutoff value selected for determining the results of a test for colorectal cancer, measured in copies of the miRNA per nanogram of stool RNA may be at least about 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3500, 4000, 4500, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000 or may be greater than about 60000, 70000, 80000, 90000, 100000, 200000, 300000, 400000, 500000, 600000, 700000, 800000, 900000, 1000000.

In particular embodiments of the methods and sequences disclosed herein, the cutoff value selected for determining the results of a test for colorectal cancer, measured in copies of the miRNA per nanogram of stool RNA may be less than about 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3500, 4000, 4500, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000 or may be less than about 60000, 70000, 80000, 90000, 100000, 200000, 300000, 400000, 500000, 600000, 700000, 800000, 900000, 1000000.

ROC analyses using SEQ ID NO:1-7 are presented in FIGS. 1B, 2B, 3B, 4B, 5B, 6B and 7B, with % specificity shown on the x axis and % sensitivity on the y axis. Abundance data for the same microRNAs is presented in FIGS. 1A, 2A, 3A, 4A, 5A, 6A, and 7A, which figures show numbers of copies of the relevant miRNA per nanogram of total RNA. It will be noted that in these figures the term “CRC” is used to denote cancers containing colorectal cancer cells and the term “normal” is used to indicate control or non-cancerous samples. In embodiments SEQ ID NOS:1, 2, 3, 4, 5, 6 and 7, either individually or collectively, may have higher expression levels in a colorectal cancer than in its adjacent normal tissue or other control tissues. In particular embodiments, at a suitable cut off level, SEQ ID NO:1 (hsa-miR-135b) may have a sensitivity and specificity for colorectal cancer of up to about 74.1% and 70.8% respectively, SEQ ID NO:2 (hsa-miR-221) may have a sensitivity and specificity for colorectal cancer of at least about 81.5% and 68.8% respectively, and SEQ ID NO:3 (hsa-miR-18a) may have a sensitivity and specificity for colorectal cancer of at least about 70.4% and 77.1% respectively. When used for the detection, diagnosis or prognosis of colorectal cancer, at a suitable cut off value SEQ ID NO:4 (has-miR-19a) may have a sensitivity of up to about 90% and a specificity of up to about 100% for colorectal cancer; at a suitable cut off value, SEQ ID NO:5 (has-miR-223) may have a sensitivity of up to about 80% and a specificity of up to about 100%; at a suitable cut off value, SEQ ID NO:6 (has-miR-301a) may have a sensitivity of up to about 70% and a specificity of up to about 100% and SEQ ID NO:7, (has-miR-592) may have a sensitivity of up to about 70% and specificity of up to about 100%.

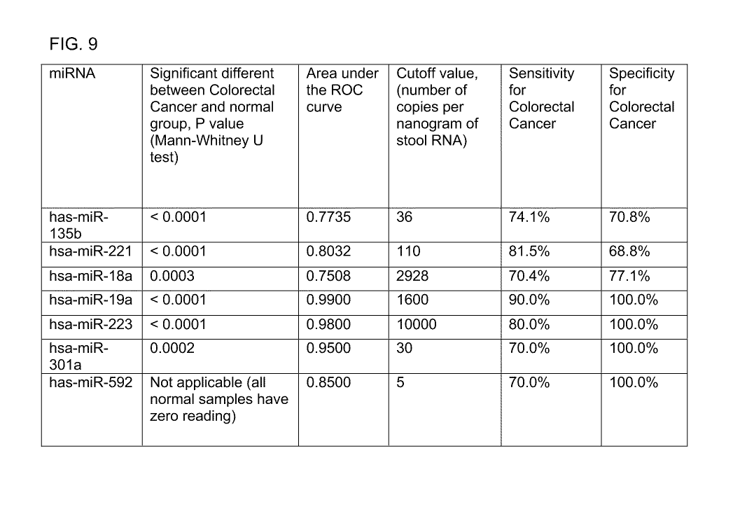

FIG. 9 shows P values for Mann-Whitney comparisons of control samples and samples comprising colorectal cancer cells. The same figure also gives AUC (area under curve), cutoff value, sensitivity and specificity dates for each of SEQ ID NOS:1-7. It will be readily understood by those skilled in the art that the cutoff values may be adjusted with consequent changes to the specificity and sensitivity of the analysis. Those skilled in the art will readily adjust such parameters to suit particular applications and requirements.

In embodiments, the materials and methods disclosed may be applied to assess the presence or prognosis of a cancer or other factors relating to the cancer. All of which will be readily understood by those skilled in the art.

### EXAMPLES

The following are examples that illustrate materials, methods, and procedures for practicing the subject matter of the embodiments disclosed. It should be understood that the examples and embodiments described herein are for illustrative purposes only and that various modifications or changes in light thereof will be suggested to persons skilled in the art and are to be included within the spirit and purview of this application.

### Methods and Materials

### Sample Categories and Handling

Fresh human stool sample is collected with a 50 ml specimen cup, stored at 4° C. before RNA extraction. Total RNA is extracted within 3 hours after defecation. Four categories of stool consistency were defined: ‘firm’ (the stool has clear-cut edges, maintains its own shape during handling but deforms with pressure), ‘soft’ (the stool has a uniform consistency but few or less apparent natural edges, it maintains its own shape but deforms with minimal handling), ‘loose’ (the stool has a semi-solid consistency and can take over the shape of the container), ‘watery’ (no solid pieces, completely liquid). Only ‘firm’, ‘soft’ and ‘loose’ stools are extracted for total RNA and further analyzed.

### MicroRNA Extraction

Stool of 0.2 to 0.3 g (wet weight) is added to 1 ml TriZol™ LS reagent in a 2 ml tube (Invitrogen™, Carlsbad, Calif., USA). Sample with firm consistency is homogenized by RNase-free pestles (USA Scientific™, Woodland, Calif., USA) to allow it completely deform. The 2 ml tube is vortexed for 30 seconds to allow the stool sample homogenize in TriZol™ LS reagent. Chloroform of 300 μl is added to the 2 ml tube. The 2 ml tube is further vortexed for 15 seconds and then centrifuged at 12,000 g for 15 min at 4° C. The upper aqueous phase is transferred to a new 2 ml tube, added with 1.5 volume of 100% ethanol and mixed thoroughly by pipetting. The total content of the 2 ml tube is transferred to an RNeasy™ Mini spin column of the miRNeasy™ Mini Kit (Qiagen™, Valencia, Calif., USA). The subsequent total RNA extraction is carried out following the manufacturer's guide. Total RNA is eluted in 50 μl nuclease free water. RNA concentration is measured by Nanodrop 2000 (Thermo™ Fisher Scientific, Wilmington, Del., USA).

### Reverse Transcription

Reverse transcription (RT) is performed using TaqMan™ microRNA Reverse Transcription Kit (Applied Biosystems™, Foster City, Calif., US) with a modified protocol. Briefly, 6 nmole of dNTPs (with dTTP), 2 unit reverse transcriptase, 1.2 unit RNase Inhibitor, 0.6 μl RT buffer, 0.6 μl TaqMan™ MicroRNA RT primer, and 3 ng to 6 ng total RNA is used in one RT reaction with a total volume of 6 μl. The thermal cycling conditions were as follows: 16° C. for 30 min, 42° C. for 30 min, 85° C. for 5 min and hold in 4° C. The RT product is added with 18 μl nuclease free water to make up to 24 μl in total volume. The amplification of specific microRNA was carried out using TaqMan™ microRNA Assays (Part Number: 442797, Applied Biosystems, Foster City, Calif., US), the assay number of the microRNA assays used for particular miRNA sequences, namely SEQ ID NOS:1-7, is given in FIG. 8. Each kit includes a primer for reverse transcription of specific microRNA, and primer/probe mixtures for qPCR of that microRNA.

### Real-Time Quantitative PCR

Real-time quantitative PCR (qPCR) of specific microRNA is performed using TaqMan™ microRNA assay (Applied Biosystems™, Foster City, Calif., US) with a modified protocol. Briefly, the reaction mix contains 6 μl TaqMan™ Universal PCR Master Mix (no AmpErase™ UNG) (Applied Biosystems™, Foster City, Calif., US), 0.3 μl microRNA TaqMan™ assay, and 2.4 μl diluted RT product and 3.3 μl nuclease free water. Real-time qPCR is carried out using Applied Biosystems™ 7500 Real-Time PCR System (Applied Biosystems, Foster City, Calif., US). Ct values were converted to absolute number of copies/ng RNA by using standard curves obtained from dilution series of known input quantities of synthetic DNA oligonucleotide (Invitrogen™, Carlsbad, Calif., USA).

### Results

Using microRNAs for Screening for Colorectal Cancer (“CRC”)

The results of screening are shown in FIG. 9 where it will be seen that SEQ ID NOS:1-7 have high specificities and sensitivities for colorectal cancer. Cutoff values and AUC values for the ROC curves for the different miRNAs are also presented in FIG. 9 along with P values form Man-Whitney U tests, indicating the significance levels of differences between cancerous samples and normal controls.

The embodiments and examples presented herein are illustrative of the general nature of the subject matter disclosed and are not limiting. It will be understood by those skilled in the art how these embodiments can be readily modified and/or adapted for various applications and in various ways without departing from the spirit and scope of the subject matter disclosed. The subject matter hereof is to be understood to include without limitation all alternative embodiments and equivalents. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed. Particular embodiments may alternatively comprise or consist of or exclude any one or more of the elements disclosed.

What is claimed is:

1. A method for diagnosing or providing a prognosis for colorectal cancer cells in a biological sample, the method comprising the steps of detecting the presence in the biological sample of an RNA sequence at least about 98% similar over the full sequence length to a sequence selected from the group consisting of SEQ ID NOS:1-7, wherein the presence of said RNA sequence is indicative of the presence of colorectal cancer cells in said sample.

2. The method according to claim 1 further comprising comparing the level of the sequence in the sample to the level of the sequence in a control.

3. The method according to claim 1 wherein the similarity is at least about 99% over the full sequence length.

4. The method according to claim 1 wherein the similarity is about 100% over the full sequence length.

5. The method according to claim 1 further comprising detecting at least two said sequences.

6. The method according to claim 1 further comprising detecting at least four said sequences.

7. The method according to claim 1 wherein the biological sample is a stool sample.

8. The method according to claim 1 wherein the group consists of SEQ ID NOS:4-7.

9. The method according to claim 1 wherein said detecting comprises amplifying the sequence.

10. A method for diagnosing or providing a prognosis for colorectal cancer in a biological sample, the method comprising detecting in the sample an RNA sequence that hybridises under high stringency conditions with a sequence selected from the group consisting of SEQ ID NOS:1-7, wherein the presence of said RNA sequence is indicative of the presence of colorectal cancer cells in the sample.

11. The method according to claim 10 further comprising comparing the level of said sequence in said sample to the level of the sequence as in control.

12. The method according to claim 10 further comprising detecting at least two of said sequences.

13. The method according to claim 12 further comprising detecting at least four said sequences.

14. The method according to claim 10 wherein the sample is a stool sample.

15. The method according to claim 10 wherein the group consists of SEQ ID NOS:4-7.

16. A kit for diagnosing or providing a prognosis for colorectal cancer cells in a biological sample, the kit comprising:
a primer suitable to reverse transcribe an RNA with at least about 98% similarity over at least 15 contiguous base pairs to any one of SEQ ID NOS:1-7.

17. The kit according to claim 16 wherein the sequence similarity is about 100% over the full length of the sequence.

18. The kit according to claim 16 wherein the biological sample is a stool sample.

19. The kit according to claim 16 wherein
said reverse transcription generates a DNA sequence and
said kit further comprises a primer pair suitable to amplify said reverse transcribed DNA sequence.

20. A probe for diagnosing or providing a prognosis for colorectal cancer in a biological sample, said probe comprising a sequence selected from the group consisting of SEQ ID NOS:1-7, wherein said sequence hybridises under high stringency conditions with a RNA sequence present in said sample, and wherein the presence of said RNA sequence is indicative of the presence of colorectal cancer cells in the sample.
